(19) United States
(12) Patent Application Publication
(10) Pub. No.: US 2007/0060796 A1
(43) Pub. Date: Mar. 15, 2007

(54) Insulin pump for use in conjunction with mobile communication terminal capable of measuring blood glucose levels and network system for transmitting control information for insulin pump

(76) Inventor: Yoon Nyun Kim, Seoul (KR)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/576,890

(22) PCT Filed: Oct. 19, 2004
(86) PCT No.: PCT/KR04/02673

§ 371 Date: Apr. 24, 2006

(30) Foreign Application Priority Data
Oct. 24, 2003 (KR) 10-2003-0074763

(51) Int. Cl. A61B 5/00

(52) U.S. Cl. 600/300; 128/904; 128/920

(57) ABSTRACT

An insulin pump for use in conjunction with a mobile communication terminal capable of measuring a blood glucose level, and a network for transmitting control information for the insulin pump are disclosed. The insulin pump includes an external input port, an output port, memory, a key input unit, a control unit, and a motor driver. The external input port receives information on the amounts of insulin to be injected. The output port outputs information on the amounts of insulin injected. The memory stores the information on the amounts of insulin injected. The key input unit inputs the status of a user. The control unit extracts the information on the amounts of insulin to be injected from the memory. The motor drive operates a soft motor to supply insulin.

### TECHNICAL FIELD

The present invention relates, in general, to the provision of control signals to an insulin pump through a mobile communication terminal and, more particularly, to an insulin pump for use in conjunction with a mobile communication terminal capable of measuring a blood glucose level, a network system for transmitting control information for the insulin pump and a method of controlling the insulin pump through the network system, in which a user's blood glucose levels are measured through the mobile communication terminal capable of measuring a blood glucose level, information on the measured glucose levels are provided to a medical server through the mobile communication terminal, information on the amounts of insulin to be injected to the user are generated by the medical server, and the insulin pump is operated by the mobile communication terminal based on the generated information on the amounts of insulin to be injected.

### BACKGROUND ART

Recently, with the development of industrial society, the number of diabetes patients is increasing due to excessive eating and stress. In general, it is reported that the number of diabetes patients has reached 10% of the total population and the number of diabetes patients in Korea has reached about 2.50 Millions. In the case of diabetes patients, the beta cells of the pancreas have a low ability to produce insulin or have lost the ability to produce insulin. Diabetes patients make efforts to adjust their blood glucose levels to a normal level through a dietary treatment or insulin injection. If a high blood glucose level is maintained for a long period, diseases attributable to diabetes, such as kidney disorders or various complications, can arise, which may jeopardize diabetes patients' lives.

Methods of managing diabetes include dietary therapy, exercise therapy and medicinal therapy. Diabetes patients must manage blood glucose levels by themselves through frequent tests and learning. Generally, diabetes patients employ medicinal therapy, along with dietary therapy and exercise therapy. The medicinal therapy artificially supplies insulin by administering an oral blood glucose reduction agent or insulin to diabetes patients' bodies at predetermined times, and employs an insulin pump.

Insulin pumps generally employ a Neutral Protamine Hagedorn (NPH) injection method that performs injection one time a day regardless of the secretion of insulin by a human body, and a Regular Insulin (RI) injection method that administers insulin at predetermined times to be in harmony with the insulin secretion curve of a human body. The NPH injection method performs injection one time a day, so that it is problematic in that a low blood glucose range occurs in the time band that does not correspond to the insulin secretion curve of a human body. The RI injection method administers insulin four or seven times a day, so that it is problematic in that side effects may be caused due to the abuse of insulin in the case where it does not match the conditions, such as the amount of food eaten by a diabetes patient and the amount of exercise taken by the diabetes patient.

The multiple amounts of insulin to be injected must be set every day, so that it is difficult for the elderly to use insulin pumps and it is inconvenient even for average patients to use such insulin pumps.

### DISCLOSURE OF THE INVENTION

Accordingly, the present invention has been made keeping in mind the above problems occurring in the prior art, and an object of the present invention is to provide an insulin pump for use in conjunction with a mobile communication terminal capable of measuring blood glucose levels and a network system for transmitting control information for the insulin pump, in which information on measured blood glucose levels is provided to a medical server through the mobile communication terminal, information on the amounts of insulin is received from the medical server, and the insulin pump is controlled based on the information on the amounts of insulin to be injected, thus not only improving the convenience of use of the insulin pump but also performing the supply of precise amounts of insulin.

In accordance with a first aspect of the present invention, the present invention provides an insulin pump for use in conjunction with a mobile communication terminal capable of measuring a blood glucose level, including an external input port connected to the mobile communication terminal, which is capable of measuring a blood glucose level and transmitting information on measured blood glucose levels, to receive information on the amounts of insulin to be injected, which corresponds to the information on the measured glucose levels, from the mobile communication terminal; an output port for outputting information on amounts of insulin actually injected to a user; memory for storing information on the amounts of insulin injected; a key input unit for inputting status before and after each meal and before retiring in electrical signal form; a control unit for extracting the information on the amounts of insulin to be injected from the memory in response to a key signal of the key input unit and generating control code according to the information on the amounts of insulin to be injected; and a motor drive for operating a soft motor to supply insulin in response to the control code.

Preferably, the external input port and the output port are USB ports or infrared ports.

Additionally, the control unit is operated in conjunction with an LCD panel for accumulating the information on the amounts of insulin injected for a predetermined period and displaying the accumulated information on the amounts of insulin injected in graphic form, and a driver for operating the LCD panel.

Additionally, the control unit accumulates the information on the measured blood glucose levels for a predetermined period, and displays the accumulated information on the measured blood glucose levels on the LCD panel with respect to a plurality of time bands and dates.

Additionally, the mobile communication terminal provides information on the amount of food eaten by the user to the blood glucose management server in coded signal form, and the information on the amounts of insulin injected is processed according to the information on the amount of food eaten.

Additionally, the key input unit includes an automatic setting mode for automatically injecting insulin and a time input mode for setting the time when insulin is to be injected, and, when the automatic setting mode is selected, the control unit controls the motor driver based on time information that is input from an internal timer and the set time when insulin is to be injected.

In accordance with a second aspect of the present invention, the present invention provides a network system for transmitting control information for an insulin pump for use in conjunction with a mobile communication terminal capable of measuring a blood glucose level, including a DB for storing information on the amounts of insulin injected that corresponds to information on measured blood glucose levels, the amount of food eaten and the amount of exercise taken; a blood glucose management server for receiving the information on the blood glucose levels that are measured by the mobile communication terminal, and the information on the amount of food eaten and the amount of exercise taken that is input via the mobile communication terminal, extracting information on the amounts of insulin to be injected that corresponds to the information on the measured blood glucose levels, the amount of food eaten and the amount of exercise taken and generating transmitter information of the mobile communication terminal; and a communication server for converting the information on the amounts of insulin to be injected into coded information on the amounts of insulin to be injected, and transmitting the coded information on the amounts of insulin to be injected to the mobile communication terminal that corresponds to the transmitter information.

Preferably, the information on the amounts of insulin injected stored in the DB is classified according to clinical histories of diabetes patients, and the blood glucose management server extracts the information on the amounts of insulin to be injected from the DB with respect to each diabetes patient based on the transmitter information of the mobile co mication terminal.

Additionally, the communication server transmits the coded information on the amounts of insulin to be injected in short message form, in conjunction with an SMS system.

Additionally, the coded information on the amounts of insulin to be injected corresponds to amounts of insulin to be injected before and after breakfast, before and after lunch, before and after dinner and before retiring, and is information on operational control of the insulin pump that corresponds to amounts of insulin to be injected with respect to insulin injection time bands.

Additionally, the mobile communication terminal is one of a mobile phone, a Personal Digital Assistant (PDA) and a Personal Computer (PC) equipped with a wireless modem, which are capable of wirelessly accessing an Internet.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a configuration diagram showing the principal functions of an insulin pump according to the present invention;

FIG. 2 is a configuration diagram showing the entire system of the present invention; and

FIG. 3 is a flowchart showing the principal operation of the entire system of the present invention.

### BEST MODE FOR CARRYING OUT THE INVENTION

Reference should now be made to the drawings, in which the same reference numerals are used throughout the different drawings to designate the same or similar components.

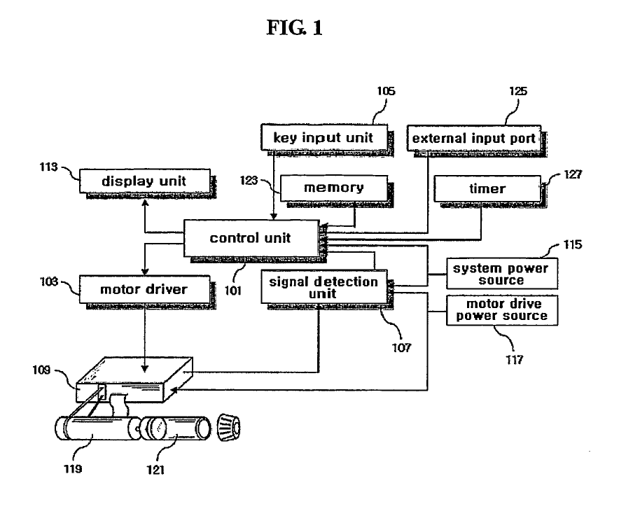

A preferred embodiment of the present invention is described in detail with reference to the accompanying drawings. FIG. 1 is a diagram showing the construction of an insulin pump according to the present invention. The insulin pump according to the present invention receives information on the amounts of insulin to be injected through the external access port of a mobile communication terminal (not shown), for example, a Universal Serial Bus (USB) port. In this case, the mobile communication terminal is equipped with the functionality of detecting a user's blood glucose level. The detailed information on the mobile communication terminal capable of measuring blood glucose levels is disclosed in Korean Pat. Appl. No. 10-2003-0073185 entitled “Mobile Communication Terminal having Blood Glucose Measurement Functionality and Medical Examination System and Method Using the Same” and filed on Oct. 20, 2003 by the present applicant. Accordingly, a detailed description of the mobile communication terminal capable of measuring a blood glucose level is omitted here.

Meanwhile, as shown in FIG. 1, the insulin pump according to the present invention includes a key input unit 105 for managing data provided to the insulin pump by the mobile communication terminal and controlling the functionality of the insulin pump, an external input port 125 connected to the mobile communication terminal to receive information on the amounts of insulin to be injected, which corresponds to information on measured blood glucose levels, from the mobile communication terminal, an output port for outputting information on the amounts of insulin actually injected into the user, memory 123 for storing information on the amounts of insulin actually injected, a control unit 101 for extracting the information on the amounts of actually injected insulin from the memory 123 in response to a key signal from the key input unit 105 and generating control code corresponding to the extracted information on the amounts of actually injected insulin, a motor driver 103 for operating the soft motor 109 of the insulin pump to supply insulin in response to the control code, a timer 127 connected to the input port of the control unit 101, and a display unit 113 connected to the output terminal of, the control unit 101 to display information on the amounts of insulin to be injected and the functions of the insulin pump.

The control unit 101 is connected to a signal detection unit 107 that is used to detect the operational status of the soft motor 109 and monitors a system power source 115 and a motor drive power source 117 of the soft motor 109. The external input port 125 and the output port may be Universal Serial Bus (USB) ports or infrared ports according the functionality of the mobile communication terminal.

The soft motor 109 controls a piston drive unit 119 that operates the piston of a cylinder 121 to load insulin, and responds to the control code provided by the control unit 101.

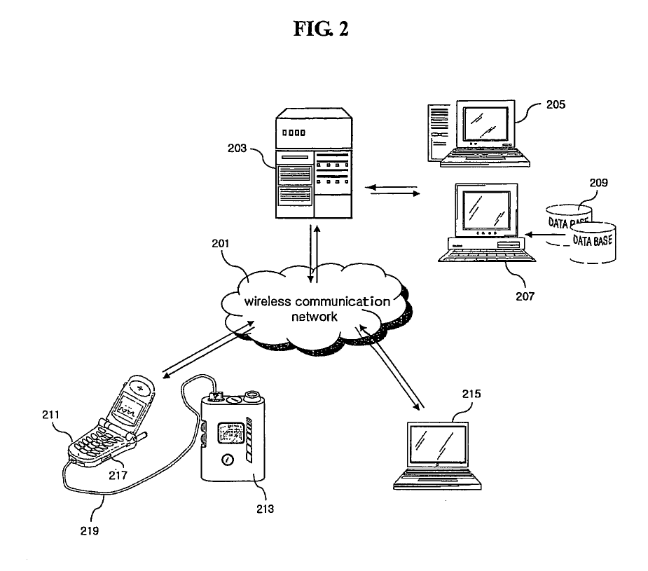

The network system is constructed as shown in FIG. 2 so that the insulin pump is connected to the mobile communication terminal and then provided with information on the amounts of insulin to be injected via a wireless communication network.

FIG. 2 is a diagram showing a network system for transmitting control information for an insulin pump 213 that is connected to a mobile communication terminal 211. As shown in FIG. 2, the network system includes the mobile communication terminal 211 for measuring blood glucose levels and transmitting information on the measured blood glucose levels, the insulin pump 213 for receiving information on the amounts of insulin to be injected from a specific port of the mobile communication terminal 211 and supplying insulin in response to the information on the amounts of insulin to be injected, a DataBase (DB) 209 for storing information on the amounts of insulin to be injected that corresponds to information on measured blood glucose levels, the amount of food eaten and the amount of exercise taken, a blood glucose management server 207 for receiving the information on the blood glucose levels measured by the mobile communication terminal 211, and information on the amount of food eaten and information on the amount of exercise taken that are input via the mobile communication terminal 211, and extracting information on the amounts of insulin to be injected that corresponds to the information on the measured blood glucose levels, the amount of food eaten and the amount of exercise taken, and a communication server 203 for converting the information on the amounts of insulin to be injected into coded information on the amounts of insulin to be injected and transmitting the coded information on the amounts of insulin to be injected to the mobile communication terminal 211 that corresponds to information on a transmitter.

The mobile communication terminal is preferably one of a mobile phone, a PDA, and a PC equipped with a wireless modem that are capable of accessing the wireless Internet.

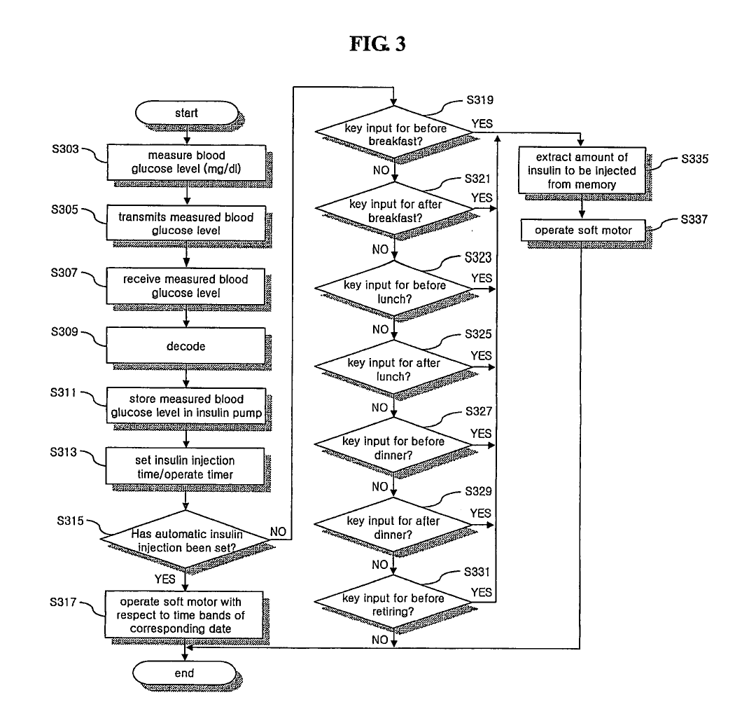

The operation of the present invention is described below. FIG. 3 is a flowchart showing the operation of the present invention. At step S303, the user measures the user's blood glucose levels through the blood glucose detection unit 217 of the mobile communication terminal 211 that is capable of measuring a blood glucose level. The blood glucose detection unit 217 can measure a blood glucose level in a blood drawing manner or non-blood drawing manner. In the case of the non-blood drawing manner, a body contact terminal is used and glucose is measured on the user's skin, while in the case of the blood drawing manner, blood is drawn using a sensor and blood glucose is measured from the drawn blood. The mobile communication terminal 211 measures the user's blood glucose levels through the blood glucose detection unit 217. The blood glucose levels are measured before and after each meal and before retiring. The information on measured blood glucose levels is stored in the specific memory of the mobile communication terminal 211.

At step S305, the user accesses the communication server 203 via the wireless communication network 201 using the mobile communication terminal 211. The communication server 203 communicates with the blood glucose management server 207 based on a Uniform Resource Locator (URL) provided by the mobile communication terminal 211.

The blood glucose management server 207 receives the terminal number information of the mobile communication terminal, searches for member information corresponding to the terminal number information, and extracts information on measured blood glucose levels, which corresponds to the member information, from the DB 209. The information on measured blood glucose levels is formed by storing information on glucose levels, which are measured for each member, for a certain period. The blood glucose management server 207 can detect the variation in blood glucose. Accordingly, the blood glucose server 207 sets the amounts of insulin to be injected into a member on the next day based on the variation in the blood glucose of the member.

That is, the blood glucose management server 207 extracts information on the amounts of insulin to be injected, which corresponds to information on the variation in blood glucose for the member, by comparing the information on the variation in blood glucose with the information on the amounts of insulin injected that is stored in the DB 209. The information on the amounts of insulin to be injected may be databased experimental information or empirical information that is determined by medical personnel.

For example, when the injection of insulin is performed five times (morning, afternoon, evening, day and night) and the measurement of blood glucose is performed seven times (before and after breakfast, before and after lunch, before and after dinner and before retiring) a day, the amounts of insulin to be injected on the next day are adjusted based on the variation of blood glucose according to the amounts of insulin injected.

TABLE 1

| | | | | | | Blood glucose level (mg/dl) | | | | | | |
|---|---|---|---|---|---|---|---|---|---|---|---|---|
| | | | | | | Morning | | Afternoon | | Evening | | Retiring |
| Amount of insulin injected (unit) | | | | | | before | after | before | after | before | after | before |
| No. | morning | afternoon | evening | day | night | meal | meal | meal | meal | meal | meal | retiring |
| 1 | 04 | 04 | 04 | 04 | 04 | 119 | 132 | 114 | 132 | 90 | 130 | 153 |
| 2 | 04 | 04 | 04 | 04 | 04 | 190 | 152 | 109 | 128 | 147 | 147 | 158 |
| 3 | 04 | 04 | 02 | 04 | 04 | 117 | — | 87 | 147 | 124 | 142 | 127 |
| 4 | 03 | 02 | 04 | 06 | 04 | 113 | 137 | 118 | 128 | 107 | 140 | 134 |
| 5 | 03 | 02 | 04 | 06 | 04 | 116 | 111 | 85 | 147 | 109 | 138 | 141 |
| 6 | 03 | 02 | 04 | 06 | 04 | 110 | 125 | 92 | 124 | 117 | 103 | 141 |
| 7 | 03 | 02 | 04 | 06 | 04 | 104 | 103 | 91 | 107 | 133 | 115 | 110 |

As described above, Table 1 contains experimental or empirical information and the information on the amounts of insulin injected is stored in the DB with respect to each member. In the blood glucose management server 207, the information on the average of the information on measured blood glucose levels with respect to each date is calculated, the variation in information on the accumulative average of the blood glucose levels provided by the mobile communication terminal 211, and information on the amount of insulin to be injected is extracted by searching for information on the average with respect to each date corresponding to the information on the calculated accumulative average.

Accordingly, the information on measured blood glucose levels with respect to each member, which is received by the blood glucose management server 207, is accumulated in the DB 209, the average of the information on measured blood glucose levels with respect to each member is calculated, and information on the amount of insulin to be injected for each time band of a corresponding date is extracted by searching information for a date having information similar to on the average of the information on measured blood glucose levels stored in the DB 209.

The blood glucose management server 207 provides the information on the amount of insulin to be injected for each time band, which is extracted from the DB 209, to a short message server 205. Accordingly, the short message server 205 converts the information on the amount of insulin to be injected for each time band into transmission data corresponding to a short message protocol through step S307, and provides the transmission data to the mobile communication terminal 211 through the communication server 203.

When an alarm message is provided to the mobile communication terminal 211 after the information on the amount of insulin to be injected for each time band has been received by the mobile communication terminal 211, the user transmits the information on the amount of insulin to be injected for each time band to the insulin pump 213 through the external port of the mobile communication terminal 211 such as a USB port or an infrared port.

In the case of using the USB port, the external port of the mobile communication terminal 211 is connected to an external access line 219, and the end terminal of the external access line 219 is connected to the external input port 125 of the insulin pump 213. In contrast, when the mobile communication terminal 211 is equipped with the infrared port and the external input port 125 of the insulin pump 213 has an infrared ray reception function, the use of the external access line 219 is unnecessary. In this case, local area communication technology using such an infrared port is well known, so that a detailed description thereof is omitted here.

At step S311, the insulin pump 213 receives the information on the amount of insulin to be injected for each time band from the mobile communication terminal 211, and the information on the amount of insulin to be injected for each time band is stored in the memory 123 under the control of the control unit 101. The information on the amount of insulin to be injected for each time band may be based on the case where insulin is injected five times a day. In this case, insulin may be injected in the morning, the afternoon, the evening, day and night. The information on the amount of insulin to be injected for each time band includes time information indicating the time band, information on the amount of insulin corresponding to the time band, and key signal information corresponding to the time information.

The control unit 101 detects that power is being normally supplied by the system power source 115, and then continuously receives time information from the timer 127. At step S313, the user sets insulin injection time. This step S313 is used to provide against the case where insulin is not injected due to the user's carelessness. The input of a key input signal is performed five times a day if the user's meal time is irregular.

Consequently, the user can set the operation of the insulin pump 213 to be automatic through the key input unit 105, and may change the operation of the insulin pump 213 to manual operation based on the variation of meal times. At step S315, when the insulin pump 213 is set to be automatically operated, the control unit 101 continuously receives current time from the timer 127, receives the time information contained in the information on the amount of insulin to be injected for each time band that is stored in the memory 123, and determines whether the current time is the time when insulin is to be injected.

If it is determined that the current time is the time when insulin is to be injected, the control unit 101 extracts information on the amount of insulin to be injected for the current time band from the memory 123, and transmits the information on the amount of insulin to be injected for the current time band to the motor driver 103. The motor driver 103 operates the soft motor 109 to correspond to the information on the amount of insulin to be injected for the current time band. The soft motor 109 injects insulin, which is loaded in the cylinder 121, into the user's body by operating the piston drive unit 119.

Meanwhile, if it is determined that insulin is set to be manually injected at step S315, the control unit 101 extracts the key signal information for each time band from the memory 123, and enables the key input unit 105. The user notifies the control unit 101 of current status before and after breakfast, before and after lunch, before and after dinner and before retiring through the key input unit 105. The control unit 101 receives a key signal indicating the current status, compares the current status with the key signal information for each time band, and extracts information on the amount of insulin to be injected that corresponds to the key signal if there exists the key signal information corresponding to the key signal.

When the insulin injection time comes, the information on the amount of insulin to be injected is transmitted to both the user's mobile communication terminal and an attending doctor, so that a diabetes patient can be stably managed. Moreover, information on the amount of insulin actually injected is stored in the database and is transmitted to the attending doctor, so that the administration of insulin can be scientifically and stably carried out.

The control unit 101 transmits information on the amount of insulin to be injected to the motor drive 103, and the motor driver 103 operates the soft motor 109 to correspond to the information on the amount of insulin to be injected.

In this, case, one of key signals indicating status before and after breakfast, before and after lunch, before and after dinner and before retiring is selected through the key input unit 105, and current status can be selected by a specific key of the key input unit 105 after corresponding status is displayed through the display unit 113 under the control of the control unit 101. Additionally, the control unit 101 accumulates the amounts of insulin injected for a predetermined period using the memory 123, so that the convenience of use and the efficiency of blood glucose management can be improved by displaying the information on the amounts of insulin, which is accumulated in the memory 123, on the display unit 113 in graphic form with respect to a time band or a date.

In the meantime, although the DB 209 is made to determine the amounts of insulin to be injected based on the information on the measured blood glucose levels, another embodiment of the present invention may allow the amounts of insulin to be injected to be determined based not only on the information on the measured blood glucose levels but also on the information on the amount of food eaten and/or information on the amount of exercise taken. For example, since the amounts of insulin to be injected must increase when the amount of food eaten and the amount of exercise taken are large, a proportional factor proportional to the amount of food eaten and the amount of exercise taken is set and the information on the amounts of insulin to be injected is processed based on the proportional factor, or the information on the amounts of insulin to be injected can be adjusted so that a basic amount of insulin can be injected in the day time or while asleep.

Industrial Applicability

As described above, the present invention provides the insulin pump for use in conjunction with the mobile communication terminal capable of measuring a blood glucose level and the network system for transmitting control information for the insulin pump, in which the user's blood glucose levels are measured by the mobile communication terminal, information on the measured blood glucose levels is provided to the blood glucose management server through a wireless network, information on the amounts of insulin to be injected corresponding to the information on the measured blood glucose levels is extracted by the blood glucose management server, and the extracted information on the amounts of insulin to be injected is provided to the insulin pump through the mobile communication terminal, so that the information on the amounts of insulin to be injected can be downloaded to the insulin pump, thus improving the convenience of use, enabling the precise supply of insulin, and allowing the efficient management of blood glucose to be carried out.

Moreover, the manual and automatic setting of the insulin pump can be performed, so that the insufficient supply of insulin due to the user's carelessness can be prevented.

Although the preferred embodiments of the present invention have been disclosed as examples of the insulin pump for use in conjunction with the mobile communication terminal capable of measuring a blood glucose level and the network system for transmitting control information for the insulin pump, those skilled in the art will appreciate that various modifications, additions and substitutions are possible, without departing from the scope and spirit of the invention as disclosed in the accompanying claims.

1. An insulin pump for use in conjunction with a mobile communication terminal capable of measuring a blood glucose level, comprising:
an external input port connected to the mobile communication terminal, which is capable of measuring a blood glucose level and transmitting information on the measured blood glucose level to a glucose management server, to receive information on amounts of insulin to be injected, which corresponds to the information on measured glucose levels, from the mobile communication terminal;
an output port for outputting information on amounts of insulin actually injected to a user;
memory for storing the information on the amounts of insulin actually injected;
a key input unit for inputting status before and after each meal and before retiring in electrical signal form;
a control unit for extracting the information on the amounts of insulin to be injected from the memory in response to a key signal of the key input unit, and generating control code according to the information on the amounts of insulin to be injected; and
a motor drive for operating a soft motor to supply insulin in response to the control code;
wherein the control unit is operated in conjunction with a Liquid Crystal Display (LCD) panel for accumulating the information on the amounts of insulin injected for a predetermined period and displaying the accumulated information on the amounts of insulin injected in graphic form, and a driver for operating the LCD panel, and accumulates the information on the measured blood glucose levels for a predetermined period, and displays the accumulated information on the measured blood glucose levels on the LCD panel with respect to a plurality of time bands and dates; and
wherein the key input unit includes an automatic setting mode for automatically injecting insulin and a time input mode for setting time when insulin is to be injected, and, when the automatic setting mode is selected, the control unit controls the motor driver based on time information that is input from an internal timer and the set time when insulin is to be injected.

2. The insulin pump according to claim 1, wherein the external input port and the output port are Universal Serial Bus (USB) ports.

3. The insulin pump according to claim 1, wherein the external input port is an infrared port.

4-5. (canceled)

6. The insulin pump according to claim 1, wherein the mobile communication terminal provides information on an amount of food eaten by the user to the blood glucose management server in coded signal form, and the information on the amounts of insulin injected is processed according to the information on the amount of food eaten.

7. (canceled)

8. A network system for transmitting control information for an insulin pump for use in conjunction with a mobile communication terminal capable of measuring a blood glucose level, comprising:
a DB for storing information on amounts of insulin injected that corresponds to information on measured blood glucose levels, an amount of food eaten and an amount of exercise taken;
a blood glucose management server for receiving the information on the blood glucose levels that are measured by the mobile communication terminal, and the information on the amount of food eaten and the amount of exercise taken that is input via the mobile communication terminal, extracting information on amounts of insulin to be injected that corresponds to the information on the measured blood glucose levels, the amount of food eaten and the amount of exercise taken and generating transmitter information of the mobile communication terminal; and
a communication server for converting the information on the amounts of insulin to be injected into coded information on the amounts of insulin to be injected, and transmitting the coded information on the amounts of insulin to be injected to the mobile communication terminal that corresponds to the transmitter information; and
wherein the coded information on the amounts of insulin to be injected corresponds to amounts of insulin to be injected before and after breakfast, before and after lunch, before and after dinner and before retiring, and is information on operational control of the insulin pump that corresponds to amounts of insulin to be injected with respect to insulin injection time bands.

9. The network system according to claim 8, wherein the information on the amounts of insulin injected stored in the DB is classified according to clinical histories of diabetes patients, and the blood glucose management server extracts the information on the amounts of insulin to be injected from the DB with respect to each diabetes patient based on the transmitter information of the mobile communication terminal.

10. The network system according to claim 8, wherein the communication server transmits the coded information on the amounts of insulin to be injected in short message form, in conjunction with a Short Message Service (SMS) system.

11. (canceled)

12. The network system according to claim 8, wherein the mobile communication terminal is one of a mobile phone, a Personal Digital Assistant (PDA) and a Personal Computer (PC) equipped with a wireless modem, which are capable of wirelessly accessing an Internet.
